(19) United States
(12) Patent Application Publication
(10) Pub. No.: US 2005/0054100 A1
(43) Pub. Date: Mar. 10, 2005

(54) Methods for fibroblast differentiation

(76) Inventors: Stephen I. Rennard, Omaha, NE (US); Xiangde Liu, Omaha, NE (US)

Correspondence Address: Moser, Patterson & Sheridan, LLP, Suite 1500, 3040 Post Oak Blvd., Houston, TX, 77056-6582, US

(21) Appl. No.: 10/658,824

(22) Filed: Sep. 8, 2003

(51) Int. Cl. C12N 5/00; C12N 5/02

(52) U.S. Cl. 435/377; 435/395; 435/384

(57) ABSTRACT

The present invention encompasses methods for culturing embryonic stem cells to produce substantially homogenous populations of fibroblasts having a stable, differentiated phenotype.

### BACKGROUND OF THE INVENTION

Stem cells are undifferentiated cells that are capable of giving rise to multiple, specialized cell types and ultimately to terminally differentiated cells. Terminally differentiated cells comprise the fully functional organs and tissues in the body and are the end product of embryonic development. Stem cells have two main characteristics. First, unlike any other cells, they are capable of dividing and differentiating into many different mature cell types within the body. Second, in the case of totipotential cells, they are also able to renew themselves so that an essentially endless supply of mature cell types can be generated when needed. The potency of a stem cell is measured by the number of different cell types it can ultimately produce. The most potent stem cell is the pluripotent stem cell which can give rise to all cell types of the body. Other stem cells exist and include multipotent stem cells which give rise to two or more different cell types.

There are numerous reports of in vitro differentiation of multipotent and pluripotent stem cells in the scientific literature. For example, embryonic stem cells derived from blastocyst and post-implantation embryos can be allowed to differentiate randomly and uncontrollably into a mixed population of terminally differentiated cells in aggregates or embryoid bodies. The terminally differentiated cells comprise various cell types including extraembryonic endoderm, spontaneously contracting muscle, nerve cells, endothelium and fibroblast-like cells. However, controlled, reproducible and stable differentiation of embryonic stem cells into fibroblasts has proven to be extremely difficult.

Thus, there exists a need for a rapid method to differentiate and isolate fibroblasts directly from stem cell cultures in vitro without undue experimentation. The present invention satisfies this need and provides related advantages as well.

### SUMMARY OF THE INVENTION

The present technology provides methods for culturing embryonic stem cells to produce substantially homogenous populations of fibroblasts having a stable, differentiated phenotype. Additionally, the present invention provides methods for culturing embryoid bodies in a three-dimensional environment, where the embryoid bodies differentiate reproducibly and stably into fibroblasts in the presence or absence of cytokines including IL-4, TGF-β1 and bFGF. Moreover, the fibroblasts, once differentiated, can be expanded in a two-dimensional monolayer culture environment and retain the differentiated phenotype. The differentiated fibroblasts are useful for, e.g., screening for growth and/or differentiation factors, determining cell markers or other phenotypic characteristics of a particular fibroblast type, and testing pharmaceutical compounds or for tissue reconstitution or regeneration.

Thus, in one embodiment, the present invention provides a method for culturing embryoid bodies from embryonic stem cells comprising obtaining embryonic stem cells; culturing the embryonic stem cells to induce formation of embryoid bodies; isolating the embryoid bodies; casting the embryoid bodies in a three-dimensional scaffolding material and a cell culture medium; and growing the embryoid bodies in the three-dimensional scaffolding material and cell culture medium. Further, embodiments of the methods of the present invention may include additionally a culturing step between the obtaining step and the culturing step that induces embryoid bodies (such as hanging drop or suspention culture).

In yet another embodiment of the present invention, there is provided a method for obtaining a population of differentiated dells from embryonic stem cells comprising obtaining embryonic stem cells; culturing the embryonic stem cells to induce formation of embryoid bodies; isolating the embryoid bodies; casting the embryoid bodies in a three-dimensional scaffolding material and a cell culture medium; growing the embryoid bodies in the three-dimensional scaffolding material and cell culture medium; inducing differentiation of the embryoid bodies to produce fibroblasts; isolating differentiated cells from the three-dimensional culture and culturing the isolated, differentiated cells in a monolayer culture.

In addition, other embodiments of the present invention include embryoid bodies cultured in a three-dimensional scaffolding environment, and fibroblasts differentiated in a three-dimensional environment and grown thereafter in a monolayer environment.

### BRIEF DESCRIPTION OF THE DRAWINGS

A more particular description of the invention, briefly summarized above, may be had by reference to the embodiments of the invention described in the present specification and illustrated in the appended drawings. It is to be noted, however, that the specification and appended drawings illustrate only certain embodiments of this invention and are, therefore, not to be considered limiting of its scope. The invention may admit to equally effective embodiments as defined by the claims.

FIG. 1 is a flow chart of one embodiment of methods according to the present invention.

FIGS. 2A through 2F show are photographs of embryoid bodies and differentiated fibroblasts in three-dimensional culture.

FIGS. 3A through 3F are photographs of differentiated fibroblasts where differentiation was induced in three-dimensional culture and the cells were subsequently transferred to two-dimensional monolayer cultures.

FIG. 4 is a photograph of immunocytochemical staining of F-actin, vimentin and pan-cytokeratin in the differentiated fibroblasts.

FIG. 5 is a graph of collagen gel contraction by control and experimental populations of mouse embryonic stem cells differentiated according to the methods of the present invention.

FIG. 6 is a bar chart showing the amount of fibronectin produced by control and experimental populations of mouse embryonic stem cells differentiated according to the methods of the present invention.

FIG. 7 is a bar chart showing the amount of PGE2 produced by control and experimental populations of mouse embryonic stem cells differentiated according to the methods of the present invention.

FIG. 8 is a graph showing the cell proliferation rate of differentiated embryonic stem cells in control and experimental populations cultured according to the methods of the present invention.

### DETAILED DESCRIPTION

Reference now will be made in detail to exemplary embodiments of the invention. While the invention will be described in conjunction with these embodiments, it is to be understood that the described embodiments are not intended to limit the invention solely and specifically to only these embodiments. On the contrary, the invention is intended to cover alternatives, modifications, and equivalents that may be included within the spirit and scope of the invention as defined by the attached claims.

The present technology encompasses methods for culturing embryonic stem cells to produce substantially homogenous populations of fibroblasts having a stable, differentiated phenotype, as well as culturing embryoid bodies in a three-dimensional environment. Such stable, homogenous populations of differentiated cells are useful in assays for screening for growth and/or differentiation factors, determining cell markers or other phenotypic characteristics of a particular fibroblast type, and testing pharmaceutical compounds or for tissue reconstitution or regeneration.

In describing the invention herein, well known scientific techniques are not described in detail. However, general methods in molecular genetics and genetic engineering are described and may be found in the current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques may be found in Current Protocols in Protein Science (J. E. Colligan et al. eds., Wiley & Sons); Current Protocols in Cell Biology (J. S. Bonifacino et al., Wiley & Sons) and Current Protocols in Immunology (J. E. Colligan et al. eds., Wiley & Sons.). Reagents, cloning vectors, and kits for genetic manipulation referred to in this disclosure are available from commercial vendors such as BioRad, Stratagene, Invitrogen, CLONTECH, and Sigma-Aldrich Co. In addition, cell culture methods are described generally in the current editions of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Tissue culture supplies and reagents are available from commercial vendors such as Gibco/BRL, Corning, Nalgene-Nunc International, Sigma Chemical Co., and ICN Biomedicals.

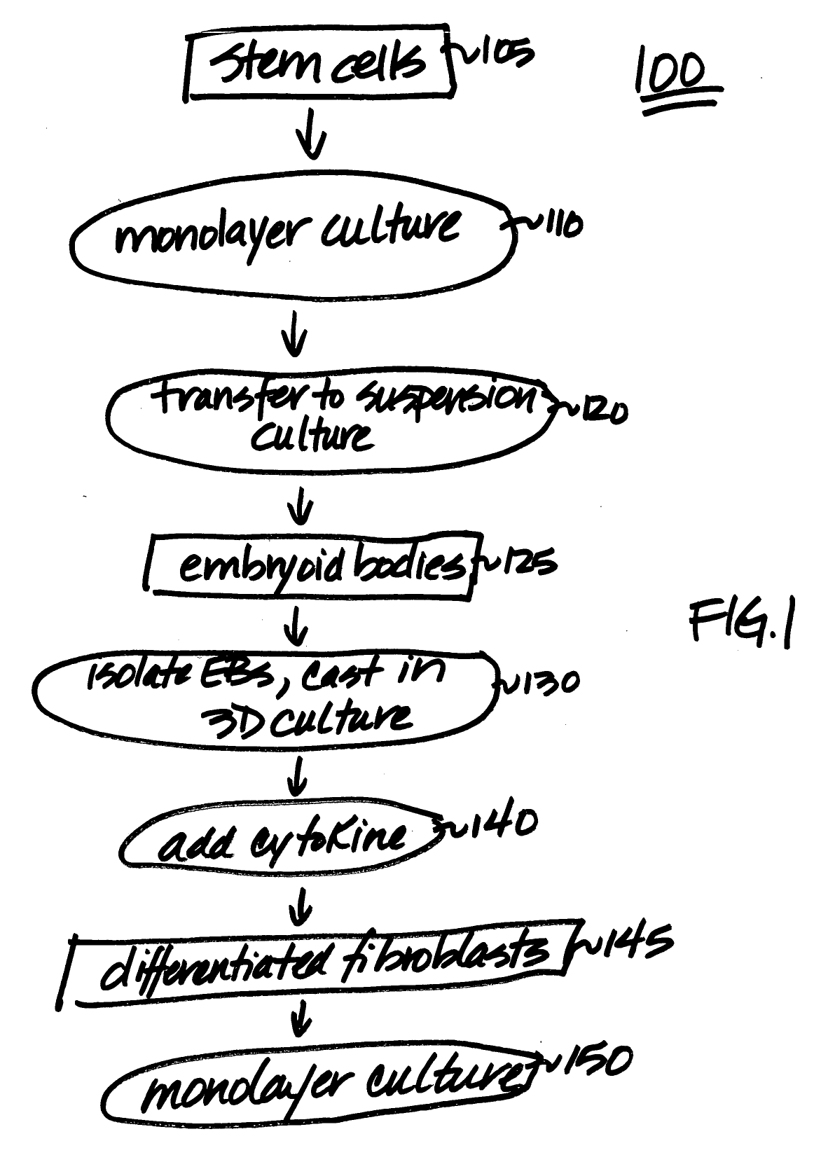

FIG. 1 shows a simplified flow chart of one embodiment of a method 100 of the present invention. At step 105 of method 100, embryonic stem cells are acquired. Suitable source cells for culturing and differentiation according to this invention include established lines of pluripotent cells derived from tissue formed after gestation. Exemplary primary tissue sources are embryonic tissue (such as a blastocyst), or fetal tissue taken any time during gestation, typically but not necessarily before 10 weeks gestation. Non-limiting exemplars are established lines of mouse embryonic stem cells, primate embryonic stem cells, and human embryonic stem cells. Alternatively, cell lines established from human bone marrow stromal cells or mesenchymal stem cells may be used (see, e.g., Liu, et al., J. Lab. Clinical Med., (2002)).

As an alternative to using established embryonic stem cell lines, embryonic stem cells may be isolated from blastocysts obtained from in vivo preimplantation embryos, in vitro fertilized embryos, or one-cell embryos expanded to the blastocyst stage (Bongso et al., Hum Reprod., 4: 706, 1989). Such embryonic stem cells can be isolated from blastocysts of mice (Evans and Kaufman, Nature, 292:154-56 (1981), members of the primate species (U.S. Pat. No. 5,843,780), and humans (U.S. Pat. No. 6,200,806).

When isolating embryonic stem cells from blastocysts, the zona pellucida is removed from the blastocysts by, e.g., brief exposure to pronase (Sigma). The inner cell masses are isolated by immunosurgery or by mechanical separation, and plated in culture. Once the inner cell mass is isolated, the cells are cultured. Techniques have been developed where mouse, primate and human embryonic stem cells may be cultured without feeder cells, yet maintain an undifferentiated phenotype (see, e.g., U.S. Pat. Pub. 20030017589). For example, primate and human embryonic stem cells may be supported in feeder-free culture on an extracellular matrix. Such a matrix can be deposited by preculturing and lysing a matrix-forming cell line (WO 99/20741), such as the STO mouse fibroblast line (ATCC Accession No. CRL-1503), or human placental fibroblasts. The extracellular matrix also can be coated directly into the culture vessel with isolated matrix components. Matrigel™ is a soluble preparation from Engelbreth-Holm-Swarm tumor cells that gels at room temperature to form a reconstituted basement membrane. Other suitable extracellular matrix components may include laminin, fibronectin, proteoglycan, entactin, heparan sulfate, and so on, alone or in various combinations.

After 9 to 15 days of culture, the inner cell mass-derived outgrowths are dissociated into clumps either by exposure to calcium and magnesium-free phosphate-buffered saline (PBS) with 1 mM EDTA, by exposure to dispase, collagenase or trypsin, or by mechanical dissociation with a micropipette. The dissociated cells are then replated in fresh medium; and observed for colony formation. Colonies demonstrating undifferentiated morphology are individually selected by micropipette, mechanically dissociated into clumps, and replated. Embryonic stem cell-like morphology is characterized as compact colonies with apparently high nucleus to cytoplasm ratio and prominent nucleoli.

The resulting embryonic stem cells are then cultured in monolayer culture (step 110 of method 100 of FIG. 1) in, for example, 80% DMEM (typically Knockout DMEM), 20% ES qualified or defined fetal bovine serum (FBS), 1% non-essential amino acids, 1 mM L-glutamine, 0.1 mM β-mercaptoethanol, leukemia inhibitory factor (LIF) (approximately 1.7 U/mL) on 0.1% gelatin-coated tissue culture plates in a 5% CO₂ incubator at about 37° C. until the desired cell density is reached. The cultures are fed every other day and split routinely by brief trypsinization, exposure to Dulbecco's PBS (without calcium or magnesium and with 2 mM EDTA), exposure to type IV collagenase (at a concentration of about 200 U/mL; Gibco) or by selection of individual colonies by micropipette.

Once a monolayer culture of embryonic stem cells has been established, the cells are transferred to a suspension culture (FIG. 1, step 120 of method 100) or a hanging drop culture to promote the formation of embryoid bodies. When embryonic stem cells are grown in suspension, they differentiate rapidly into embryoid bodies having many recognizable cell types. To establish a typical suspension culture, embryonic stem cell monolayer cultures are trypsinized, washed in medium and pelleted. The embryonic stem cells are then resuspended in medium and counted. Next, a single-cell suspension of approximatedly 6000 embryonic stem cells is seeded onto a bacterial-grade non-tissue culture dish (such as a petri dish) in a small volume of medium. The plates are placed CO₂ incubator, typically within a larger dish with open plates of water (to avoid the drying out of the cultures), re-fed daily, and cultured until there are enough embryoid bodies for induction.

Alternatively, the embryoid bodies may be cultured by the hanging drop method. In the hanging drop method, the embryonic stem cells are diluted to approximately 5-50 cells per 20 μL medium. Individual drops of 20 μL are then placed carefully onto the surface of a petri dish lid, with each drop remaining separate. The lid is placed on the plate and the plate/lid assembly is inverted, such that the drops are hanging from the top of the plate. As with the suspension cultures, these plates typically are placed in a larger dish with open dishes of water and cultured for 24 to 48 hours. The plates are then re-inverted and the dishes are filled with medium. The cultures are continued until there are enough embryoid bodies for induction.

Once there are enough embryoid bodies in the suspension or hanging drop culture to proceed (FIG. 1, 125 of method 100), the embryoid bodies are isolated by, for example, centrifugation, and cast in a three-dimensional culture environment (step 130 of method 100). The three-dimensional culture environment includes a scaffolding or matrix material, including but not limited to albumin, collagen, gelatin, hyaluronic acid, starch, alginate, pectin, cellulose and cellulose derivatives (such as methylcellulose, hydroxypropylcellulose, hydroxypropylmethylcellulose, carboxy-methylcellulose, cellulose acetate phthalate, cellulose acetate succinate, hydroxypropylmethylcellulose phthalate), casein, dextran, polysaccharides (such as sucrose acetate isobutyrate), or fibrinogen. The concentration of scaffolding material used will vary depending on the material chosen, but typically is in a concentration of about 0.2 mg/ml to about 5.0 mg/ml.

Thus, the three-dimensional culture environment includes culture medium, for example the DMEM medium as described herein, with, e.g., collagen added at an approximate concentration of 0.5 to 2 mg/ml. Type I collagen can be isolated from bovine skin, which is the major source for commercially available type I collagen. Also, Vitrogen (COHESION CO., Palo Alto, Calif.) is a commercially available type I collagen and is widely used to coat culture plate or making collagen gels. Once the type I collagen is polymerized to form gels containing embryoid bodies, medium supplemented with a low percentage of serum and cytokines or growth factors are added the gels to induce differentiation (step 140 of method 100). As is known in the art, cytokines and growth factors are a varied group of proteins released by mammalian cells that have autocrine or paracrine activity. Cytokines and growth factors are known, for example, to control cell proliferation and differentiation, to regulate immune responses, and to affect hemopoiesis and inflammatory responses.

Various cytokines and growth factors are used to induce differentiation. Such cytokines and growth factors include, but are not limited to, vascular endothelial growth factor (VEGF); vascular permeability factor (VPF); members of the fibroblast growth factor family, including acidic fibroblast growth factor (aFGF) and basic fibroblast growth factor (bFGF); the interleukins such as interleukin-1 (IL-1α, and -1β, -2, -3, -4, -5, -6, -7, -8, -9, -10, -11, -12, -13, -14, -15, -16, -17 and -18); epidermal growth factor (EGF); platelet-derived growth factor (PDGF) or platelet-derived endothelial cell growth factor (PD-ECGF); transforming growth factors alpha and beta (TGF-α, TGF-β); tumor necrosis factor alpha (TNF-α); hepatocyte growth factor (HGF); granulocyte-macrophage colony stimulating factor (GM-CSF); angiogenin; angiotropin; fibrin, nicotinamide; macrophage inflammatory protein (MIP); macrophage migration inhibiting factor (MIF); granulocyte stimulating factor (G-CSF); macrophage stimulating factor (M-CSF); endothelial cell growth factor (ECGF); members of the interferon family (IFNs); members of the insulin-like growth factor family (IGF-I and IGF-II); nerve growth factor (NGF); members of the neurotrophin family (NTs); members of the selectin family; intercellular adhesion molecule (ICAM); platelet vascular cell adhesion molecule (PECAM); vascular cell adhesion moleculre (VCAM); calcitonin, hirudin, other mediators, or hormones such as glucocorticoids.

The embryoid bodies, which are embedded inside collagen gels, are cultured, e.g., in a CO₂ incubator at 37° C. and re-fed every 2-3 days with medium containing a low percentage of serum and cytokines or growth factors until differentiated fibroblasts are obtained (usually, about 4 weeks, 145 of method 100 in FIG. 1). Various cytokines will induce different types of fibroblasts. Generally, fibroblasts are flattened, irregular, branched, motile cells found throughout connective tissue. Fibroblasts are cells of mesodermal origin that make the collagens, reticular and elastic fibers, glycosaminoglycans and glycoproteins found in the extracellular matrix of connective tissues. Fibroblasts themselves are known to be multipotent in that they give rise to other cells of mesodermal origin such as adipose cells, osteocytes, smooth muscle cells and cartilage.

Once differentiated fibroblasts appear (after about 4 weeks), the collagen gels are digested with collagenase, which releases the fibroblasts together with the embryoid bodies. The fibroblasts and embyoid bodies are then isolated by, e.g., centrifugation, and plated in serum-supplemented medium in monolayer cultures (step 150 of method 100, FIG. 1). When the fibroblasts grow to near confluence, they are separated from the embryoid bodies by, for example, trypsinization, where the fibroblasts detach from the tissue culture plates, while embryoid bodies remain attached to the plates. Once the fibroblasts have differentiated and been placed in monolayer cultures, it is not necessary to include the inducing cytokine or growth factor in the culture medium. In fact, the fibroblasts will remain stably differentiated and can be passaged many times under monolayer culture conditions. In addition, the differentiated fibroblasts can be hypocrised and kept in liquid nitrogen tank to be used for a variety of purposes.

The phenotype of the differentiated fibroblasts may be characterized by any means known in the art. Such phenotypic characterization may include but is not limited to cell morphology; detection of cell surface markers such as vimentin (a marker for mesenchymal cells) and cytokeratin (a marker for epithelial cells); ability to produce specific proteins or other biomolecules such as fibronectin or prostaglandin E2; transcription profile analysis by, for example, microarrays; and biophysical analyses such as mobility and contractility.

For culturing or characterizing cells differentiated by the methods according to the present invention, it may be desirable for the cells to be altered genetically, either in a transient or stable fashion, to give them desired properties. The genetic alteration may be done so as to give the cells a desired property in the undifferentiated state, to give them desired properties after differentiation into other cell types, or to provide a method to positively or negatively select for particular undifferentiated or differentiated phenotypes.

For therapeutic applications using the differentiated cells of the present invention, it may be beneficial to modify the cells with therapeutic genes, or to render the cells histocompatible with the intended recipient. Genetic alteration also can be used to prepare cells for sorting after differentiation. For example, cells may be transfected with a drug susceptibility gene, such as herpes simplex virus thymidine kinase (which renders cells susceptible to ganciclovir), under control of a promoter specific for undifferentiated cells, such as the OCT-4 promoter or the hTERT promoter (WO 02/42445). After the culture has been differentiated, any residual undifferentiated cells can be eliminated from the population using ganciclovir. Suitable vector plasmids for transfecting embryonic stem cells include lipid/DNA complexes, such as those described in U.S. Pat. Nos. 5,578,475; 6,020,202; and 6,051,429.

Genetic alteration of embryonic stem cells requires achieving a high efficiency of genetic alteration, while not promoting differentiation of the embryonic stem cells along an undesired pathway. Efficiencies of genetic alteration are rarely 100%, and it is usually desirable to enrich the population for cells that have been successfully altered. The genetically altered cells can be enriched by taking advantage of a functional feature of the new genotype. For example, where the embryonic stem cells are transfected with a label such as GFP, or with an immunostainable surface marker such as NCAM, then the embryonic stem cells can be suspended, separated by fluorescence-activated cell sorting, and replated.

A particularly effective way of enriching genetically altered cells is positive selection using resistance to a drug such as neomycin. To accomplish this, the cells can be genetically altered by contacting simultaneously with vector systems for the marker gene or gene of interest, and a vector system that provides the drug resistance gene. If the proportion of drug resistance gene in the mixture is low (say, 3:1), then most drug resistant cells should also contain the gene of interest. Alternatively, the drug resistance gene can be built into the same vector as the gene of interest. After transfection has taken place, the cultures are treated with the corresponding drug, and untranstected cells are eliminated.

Following genetic alteration and drug selection, it is possible to pick colonies that demonstrate the altered phenotype, and culture them separately. The picked colonies are dispersed into small clumps of 25-100 cells, and replated in a suitable environment. It is possible to achieve cultures of embryonic stem cells in which a high proportion (up to 90%) of the cells are genetically altered.

In another aspect of the present invention, the differentiated fibroblasts may be used to screen for factors (such as small molecule drugs, peptides, polynucleotides, and the like) or conditions (such as culture conditions or manipulations) that affect the characteristics of fibroblasts in culture. The culture methods of the present invention have the advantage of allowing manipulation of the cells in a monolayer culture subsequent to differentiation, and is not complicated by the need to include the initially-differentiating cytokine in the culture conditions. Thus, in one aspect, the differentiated fibroblasts of the present invention are used to test growth affecting substances in a simple medium, such as Knockout DMEM. Different wells are then treated with different cocktails of soluble factors that are candidate growth affecting substances. Efficacy of each mixture is determined by examining the treated cells for culture maintenance, retarded growth, proliferation, abnormal proliferation, etc. Likewise, potential differentiation factors or conditions can be tested by treating the cells according to the test protocol, and then determining whether the treated cell develops functional or phenotypic characteristics of a further differentiated cell, or of a de-differentiated cell.

In yet another aspect, the differentiated fibroblasts of the present invention also can be used for the testing of pharmaceutical compounds in drug research. Assessment of the activity of candidate pharmaceutical compounds generally involves combining the differentiated cells of this invention with the candidate compound, determining any resulting change, and then correlating the effect of the compound with the observed change. The screening may be done, for example, either because the compound is designed to have a pharmacological effect on certain cell types, or because a compound designed to have effects elsewhere may have unintended side effects. Two or more drugs can be tested in combination (by combining with the cells either simultaneously or sequentially), to detect possible drug-drug interaction effects. In some applications, compounds are screened initially for potential toxicity (see, e.g., Castell et al., In vitro Methods in Pharmaceutical Research, Academic Press, p. 375-410 (1997)).

In another aspect of the invention, the differentiated fibroblasts can be used to test the cytotoxicity of a test compound. Cytotoxicity of a pharmaceutical compound can be determined by the effect on cell viability, survival, morphology, on the expression or release of certain cell markers, receptors or enzymes, on DNA synthesis or repair (measured by ³H-thymidine or BrdU incorporation), or on sister chromatid exchange (determined by metaphase spread, such as disclosed in In vitro Methods in Pharmaceutical Research, Academic Press, (1997), and U.S. Pat. No. 5,030,015).

In yet another aspect, the fibroblasts differentiated by the methods according to the present invention also can be used for tissue reconstitution or regeneration in a human patient in need thereof. For therapeutic use, it is usually desirable that differentiated cell populations be substantially free of undifferentiated embryonic stem cells. The cells are administered in a manner that permits them to graft to the intended tissue site and reconstitute or regenerate the functionally deficient area. For example, irregularities in epidermal surfaces, such as skin marred by a wound or ulceration is a target of differentiated fibroblast implantation.

For purposes of commercial distribution, cells differentiated by the methods according to this invention are typically supplied in the form of a pharmaceutical composition comprising an isotonic excipient, and prepared under conditions that are sufficiently sterile for in vivo administration. For general principles in medicinal formulation of cell compositions, see Cell Therapy: Stem Cell Transplantation, Gene Therapy, and Cellular Immunotherapy, by G. Morstyn & W. Sheridan, eds, Cambridge University Press, (1996). The cells may be packaged in a device or container suitable for distribution or clinical use, optionally accompanied by information relating to use of the cells in tissue regeneration, or restoring a therapeutically important metabolic function.

The examples that follow are provided by way of further illustration, and are not meant to imply any limitation in the practice of the claimed invention.

### EXAMPLES

Monolayer culture of embryonic stem cells: Mouse embryonic stem cells from were cultured in tissue culture dishes coated with 0.1% gelatin, in Complete Media (400 mL Knockout DMEM (Gibco), 6 mL GlutaMAX-1 supplement (Gibco), 6 mL penicillin/streptomycin (Gibco), 100 mL of ES qualified fetal bovine serum (Gibco), 4.7 mL β-mercaptoethanol (Sigma M-7522) and 1000U leukemia inhibitory factor (Chemi)). The cells were incubated at 37° C. in a 5% CO₂ atmosphere, fed every 1 to 2 days and were split 1:3 at confluence.

Formation of embryoid bodies and induction of differentiation: To form embryoid bodies, the embryonic stem cells from the monolayer culture were cultured in suspension in 10 mL Basal Media (400 mL Knockout DMEM (Gibco), 6 mL GlutaMAX-1 supplement (Gibco), 6 mL penicillin/streptomycin (Gibco), 100 mL of ES qualified fetal bovine serum (Gibco)). The cells were fed every day and cultured for 3 to 5 days to obtain enough embryoid bodies for induction.

To induce differentiation, 5 mL out of 10 mL of the 7-day-old embryoid body culture was removed and spun to pellet the embryoid body cells. The pellet was mixed with 2.5 mL of 4× Knockout DMEM (Gibco), 5 mL water, and 5.5 mL rat tail tendon collagen (RTTC, extracted from rat tail tendon). One mL of the collagen-containing cell mixture was plated in each well of a 12-well tissue culture plate. After polymerization, Basal Media (described above) plus the cytokine (IL-4, TGF-β1, bFGF), with 2% ES qualified fetal bovine serum (instead of 20% ES qualified fetal bovine serum used in the monolayer culture of the embryonic stem cells) was added to the top of the cultures, and the embryoid bodies were cultured for approximately 4 weeks.

Extraction from three dimensional matrix and separation of differentiated cells from embryoid bodies: Once differentiation had taken place, the differentiated cells and embryoid bodies were dissociated from the collagen matrix by harvesting the cell-containing collagen matrix into 50 mL conical tubes. One mL of collagenase I (Sigma, 1 mg/mL) was added to each tube, and the tubes were incubated at 37° C. for 30-60 minutes or until the gels were digested. The cells were then pelleted by centrifugation at 500 g for 5 minutes.

The cell pellets were resuspended in Basal Media containing 10% ES qualified fetal bovine serum and plated into 6-well tissue culture plates (or 60 mm plates). The cells were re-fed with Basal Media containing 10% ES qualified fetal bovine serum every 2-3 days. Confluent cultures were passaged up to 5 times, or were grown to confluence, harvested, and frozen in liquid nitrogen. The differentiated fibroblasts were separated from what remained of the embryoid bodies by trypsinizing the cells. When this was done, the fibroblasts detached from the plate while the embryoid bodies remained attached to the plate. In addition, after the cell-containing collagen matrix was removed from the 12-well plates, differentiated fibroblasts were often observed to be adhered to the bottom of the well. Fresh Basal Medium containing 10% ES qualified fetal bovine serum was added to the wells containing these adherent fibroblasts and the cells were grown to confluence.

Experiments with cytokines: The differentiation of fibroblasts after treatment with three different cytokines: transforming growth factor β1 (TGF-β1), basic fibroblast growth factor (bFGF), and murine recombinant interleukin 4 (mrIL-4) was examined by several methods. TGF-β1 is a multifunctional peptide that is known to be involved with cell proliferation, differentiation, and other functions in many cell types. TGF-β1 is produced by fibroblasts, platelets, monocytes, chondrocytes and osteoblasts. FGFs are any of various growth factors with strong mitogenic potential, and are characterized by their high affinity to heparin. Interleukin 4, also known as B-cell stimulatory factor 1 or lymphocyte stimulatory factor 1, is an interleukin that participates in several B-cell activation processes. IL-4 is a co-stimulator of DNA synthesis and is a glycoprotein.

In the experiment, 5 mL out of 10 mL of a 7-day old embryoid body culture was removed and spun to pellet the embryoid body cells. The pellet was mixed with 2.5 mL of 4× Knockout DMEM (Gibco), 5 mL water, and 5.5 mL native type I collagen (RTTC). After the gels were polymerized, 5 ng/mL TGF-β1, 25 ng/mL bFGF, or 20 ng/mL mrIL-4 was applied to the culture medium. The control culture was not treated with a cytokine. One mL of the control or experimental cell mixture was plated in each well of a 12-well tissue culture plate. After attachment, the medium was changed to Basal Media (described above) containing the appropriate cytokine and 2% ES qualified fetal bovine serum (again, instead of 20% ES qualified fetal bovine serum). The cells were re-fed with fresh medium and after 8-14 days differentiated fibroblasts were seen growing outwardly from the embryoid bodies.

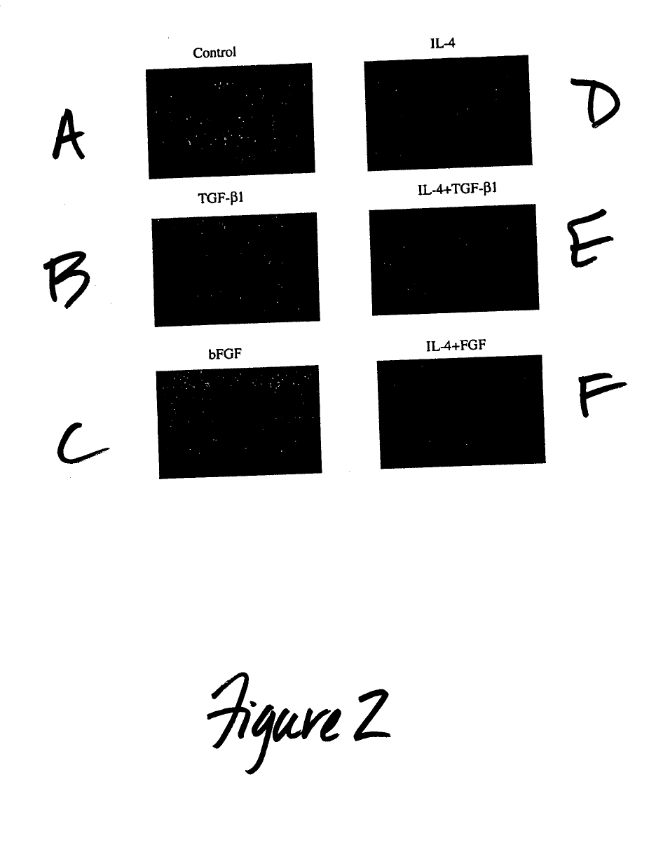

FIGS. 2A through 2F are photomicrographs (400× magnification) of the embryoid body/fibroblast cultures at day 11. Panel 2A is a photomicrograph of an embryoid body (EB) and fibroblasts (F) from the control culture. Note that the progression of differentiation of the fibroblasts is not extensive. Panel 2B is a photomicrograph of an embryoid body (EB) and fibroblasts (F) from the culture treated with cytokine TGF-β1. Note that, in comparison to Panel A, the disintegration of the embryoid body is less advanced, and the differentiation and growth of spindle-shaped differentiated fibroblasts also is less advanced than the control culture.

Panel 2C is a photomicrograph showing two embryoid bodies (EB) and fibroblasts (F) from the culture treated with cytokine bFGF. Here, the disintegration of the embryoid bodies is advanced, and the differentiation and growth of spindle-shaped fibroblasts is extensive. Panel 2D is a photomicrograph showing an embryoid bodies (EB) and fibroblasts (F) from the culture treated with cytokine mrIL-4. The growth of spindle-shaped differentiated fibroblasts is about the same as that seen in Panel A (control).

Panel 2E is a photomicrograph showing an embryoid body (EB) and fibroblasts (F) from the culture treated with cytokines TGF-β1 and IL-4, and panel 2F is a photomicrograph showing an embryoid body (EB) and fibroblasts (F) from the culture treated with cytokines FGF and IL-4. The disintegration of the embryoid body is more advanced, and the differentiation and growth of the differentiated fibroblasts is more extensive in 2F. Thus, it was observed that bFGF treated cells differentiated most quickly, growing outwardly from the embryoid bodies at about 7 days. IL-4 treated and control cells differentiated second most quickly and at about the same rate. TGF-b1 treated cells were the slowest to differentiate, taking about 2 weeks or longer to see fibroblast-like cells.

After approximately one month in culture, the collagen gels containing cells were treated with collagenase (1 mg/mL, 1 ml/gel) for 1 hour, spun and resuspended in Basal Media with 10% ES qualified fetal bovine serum. The cells were then plated into 6-well tissue culture plates pre-coated with V30 (1% Vitrogen®100 in PBS, COHESION CO., Palo Alto, Calif.) and were cultured. Differentiated fibroblasts from the three-dimensional culture that had adhered to the plate were fed with Basal Media with 10% ES qualified fetal bovine serum and allowed to continue to grow.

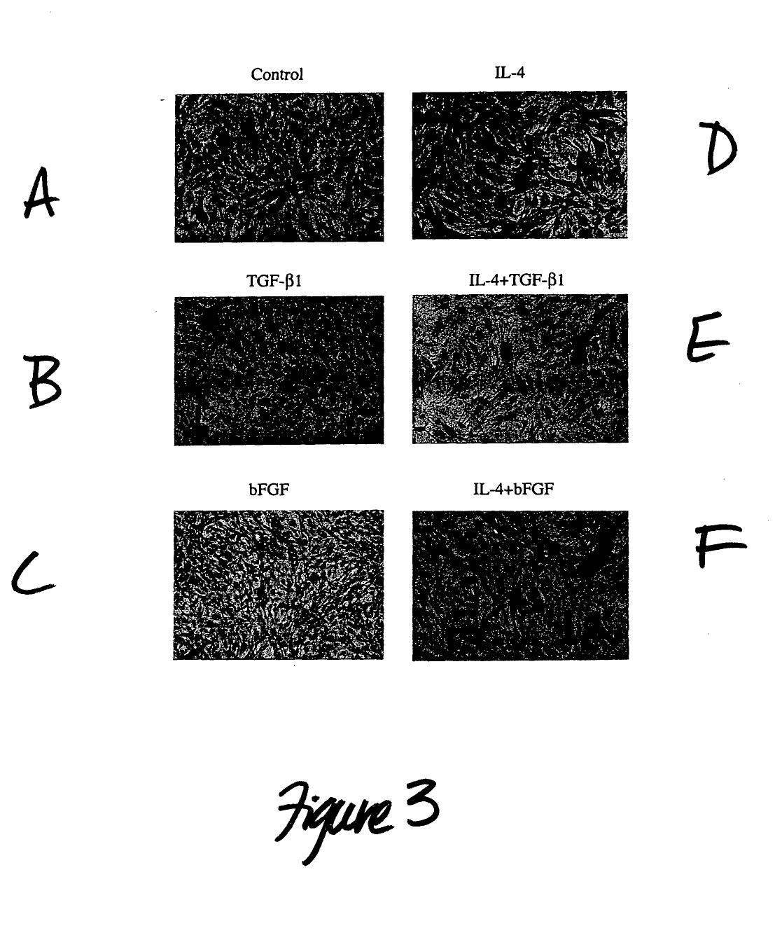

The photomicrographs in FIG. 3 were taken of the differentiated cells after two passages in two-dimensional monolayer culture at day 14. The cells were stained with Diff Quick Stain (PROTOCOL™, Fischer Scientific Co.). Panel 3A is a photomicrograph of fibroblasts from the two-dimensional monolayer control culture after differentiation, panel 3B is a photomicrograph of fibroblasts from the two-dimensional monolayer culture after treatment with cytokine TGF-β1, panel 3C is a photomicrograph of fibroblasts from the two-dimensional monolayer culture after treatment with cytokine bFGF, panel 3D is a photomicrograph of fibroblasts from the two-dimensional monolayer culture after treatment with cytokine mrIL-4, panel 3E is a photomicrograph of fibroblasts from the two-dimensional monolayer culture after treatment with cytokines TGF-β1 and IL-4, and panel 3F is a photomicrograph of fibroblasts from the two-dimensional monolayer culture after treatment with cytokines FGF and IL-4. Note that cells grown in the presence if IL-4 are large with a cubic morphology, while TGF-b1 treated cells are flat and smaller in size compared to control cells. bFGF treated cells have a small nucleus and cytoplasm.

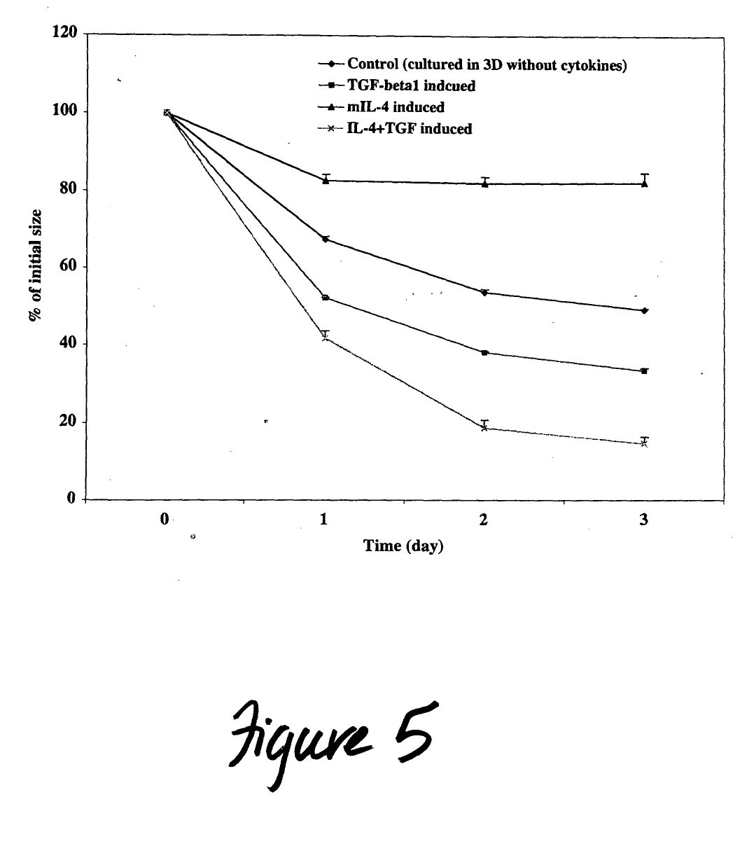

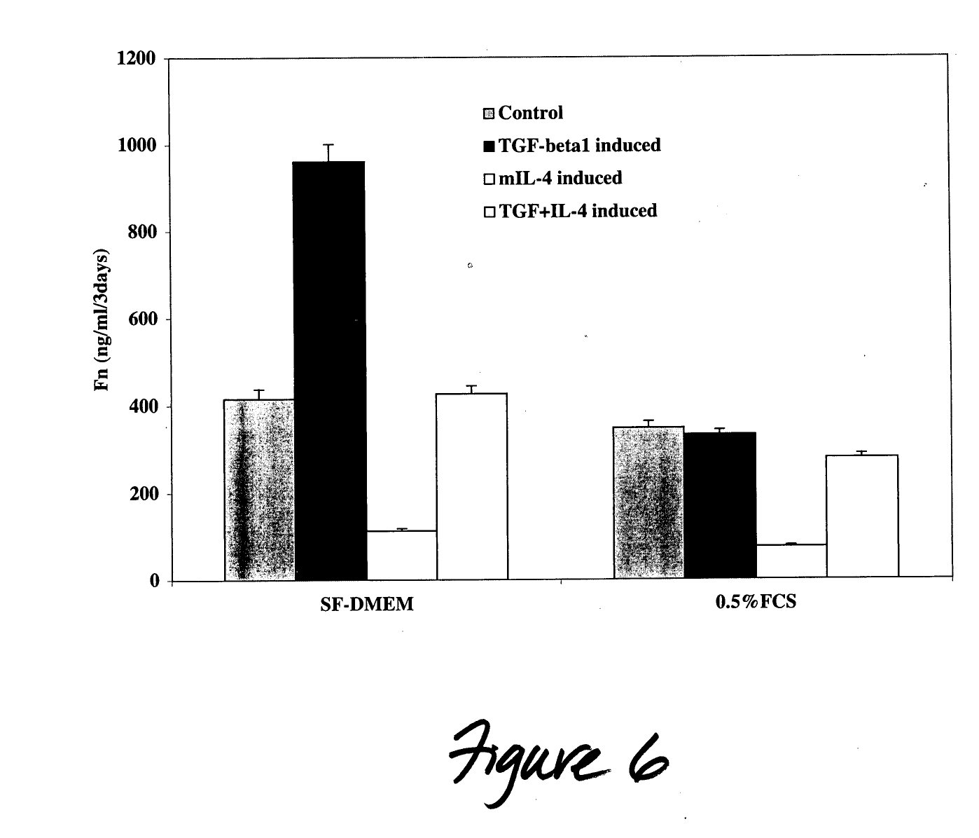

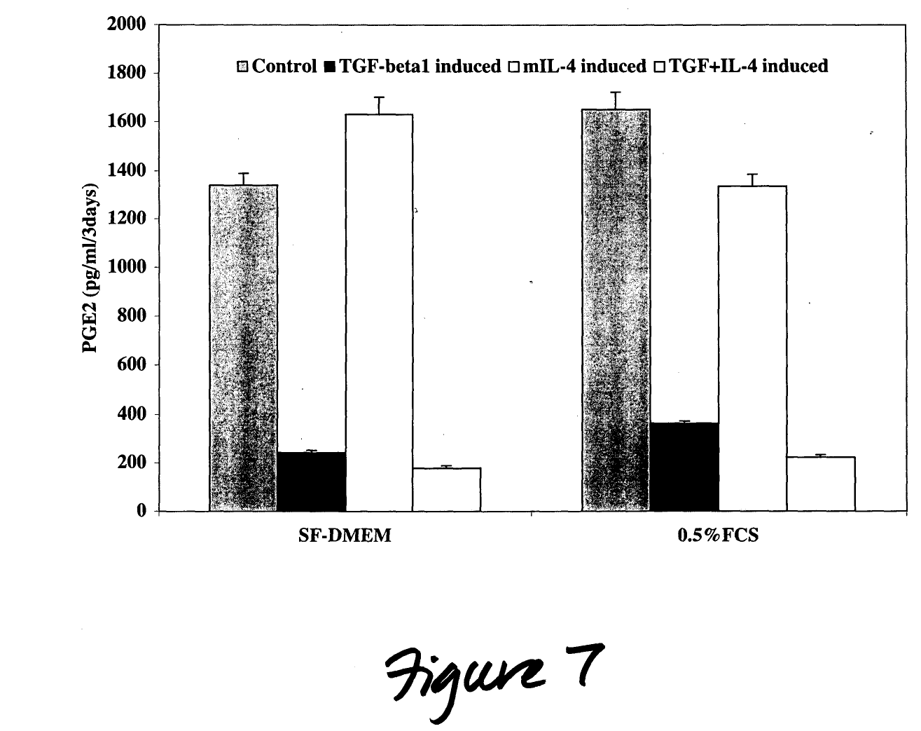

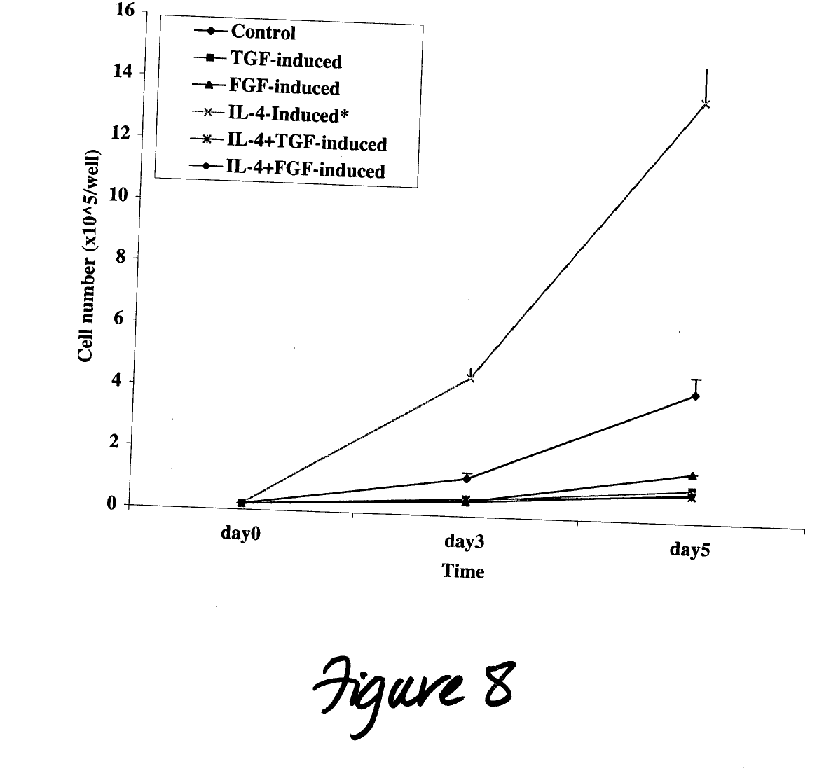

The differentiated fibroblasts were further characterized by cellular biological function such as collagen gel contraction (FIG. 5), fibronectin production (FIG. 6), prostaglandin E2 (PGE2) production (FIG. 7), and proliferation (FIG. 8).

Collagen gel contraction is an in vitro model of wound healing and tissue remodeling, and thus is widely used to study fibroblast function of tissue remodeling. The collagen gel contraction assay was accomplished by mixing fibroblasts with native type I collagen (again, Rat Tail Tendon Collagen, RTTC) to form a lattice. The collagen/fibroblast mixture was cultured in serum free DMEM for 3 days, allowing contraction of the lattice. Fibroblasts with different phenotypes tend to contract the collagen lattice differently. For example, fibroblasts from fibrotic tissue contract the collagen gel more quickly and strongly than fibroblasts from emphysema patients.

The collagen gel contraction assay was performed by casting fibroblasts into native type I collagen gels and released into serum DMEM. Gels were allowed to contract at 37° C. in 5% CO₂ atmosphere for 3 days. Gel size was measured with an image analyzer daily. Gel size was expressed as percentage of initial size.

Fibroblasts also produce a variety of substances that play important roles in tissue remodeling through autocrine or paracrine mechanisms, including fibronectin (Fn) and prostaglandin E2 (PGE2). Fibronectin and PGE2 are important regulators of collagen gel contraction and cell migration. Fn enhances collagen gel contraction and cell migration while PGE2 inhibits collagen gel contraction and cell migration. TGF-β1 induced fibroblasts produceed more Fn and less PGE2 compared to the control; thus TGF-β1 induced fibroblasts contracted the collagen gels more than the control cells did. In contrast, fibroblasts induced by IL-4 produce less Fn but more PGE2, thus the IL-4 induced fibroblasts contracted the collagen gels less than the control cells did.

Fibronectin production was quantified by ELISA, which is specific to human fibronectin. PGE2 production was quantified using a commercially available EIA kit (Cayman Chemical Co.).

Cell proliferation was assessed by plating 10⁴ cells/well in 12-well tissue culture plates and culturing the cells in 10% FCS-DMEM for 5 days. Cell number was counted by Coulter Counter on day 1, day 3 and day 5. The observed speed of cell proliferation was the following (from fastest to slowest): IL-4 induced fibroblasts, control, and TGF-β1 induced fibroblasts.

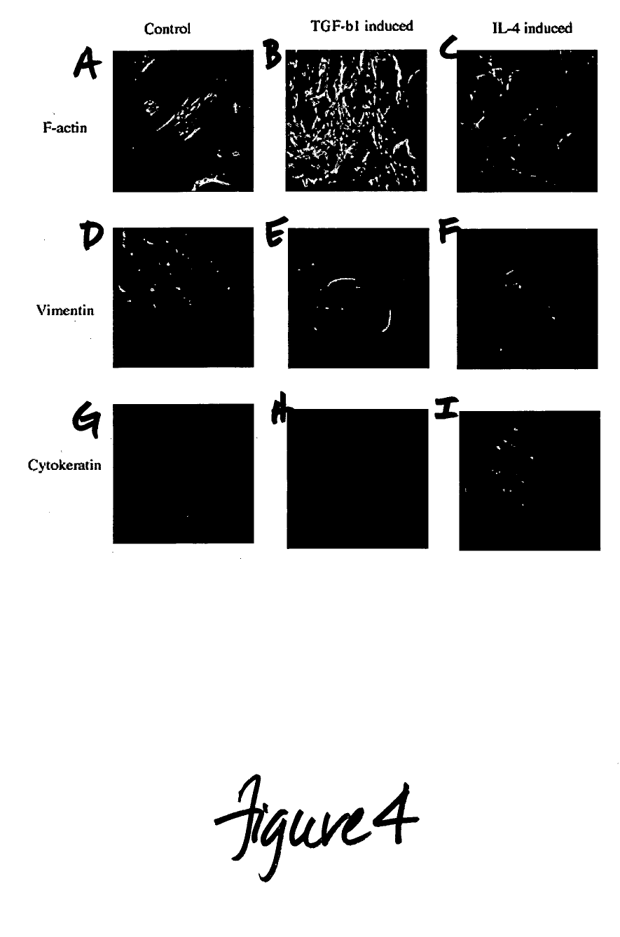

Vimentin is a marker for mesenchymal cells including fibroblasts, while cytokeratin is the marker for epithelial cells. Vimentin and cytokeratin staining with such compounds is used widely to characterize cell phenotypes. As shown in FIG. 4, the differentiated cells are vimentin positive but negative to cytokeratin staining, indicating that these cells are mesenchymal fibroblasts not epithelial cells. F-actin, a microfilament in cytoplasm, is the machinery structure that is responsible for cell contraction and migration. As is seen in FIG. 4, F-actin is strongly expressed in these differentiated fibroblasts.

While the present invention has been described with reference to specific embodiments, it should be understood by those skilled in the art that various changes may be made and equivalents may be substituted without departing from the true spirit and scope of the invention. In addition, many modifications may be made to adapt a particular situation, material, or process to the objective, spirit and scope of the present invention. All such modifications are intended to be within the scope of the invention.

All references cited herein are to aid in the understanding of the invention, and are incorporated in their entireties for all purposes.

1. A method for culturing embryoid bodies from embryonic stem cells comprising:
obtaining embryonic stem cells;
culturing the embryonic stem cells to induce formation of embryoid bodies;
isolating the embryoid bodies;
casting the embryoid bodies in a three-dimensional scaffolding material and a cell culture medium; and
growing the embryoid bodies in the three-dimensional scaffolding material and cell culture medium.

2. The method of claim 1 further comprising an additional culturing step between the obtaining step and the culturing step, wherein the additional culturing step comprises culturing the embryonic stem cells in a monolayer culture.

3. The method of claim 2, wherein the monolayer culture is performed in a culture medium of knock out DMEM and about 20% ES qualified fetal bovine serum.

4. The method of claim 1, where the culturing step is performed by suspension culture or by hanging drop culture.

5. The method of claim 4, wherein the suspension culture or hanging drop culture is performed in a culture medium of knock out DMEM and about 20% ES qualified fetal bovine serum.

6. The method of claim 1, wherein the isolating step is performed by centrifugation.

7. The method of claim 1, wherein the three-dimensional scaffolding material is albumin, collagen, gelatin, hyaluronic acid, starch, alginate, pectin, cellulose or cellulose derivatives (such as methylcellulose, hydroxypropylcellulose, hydroxypropylmethylcellulose, carboxy-methylcellulose, cellulose acetate phthalate, cellulose acetate succinate, hydroxypropylmethylcellulose phthalate), casein, dextran, polysaccharides (such as sucrose acetate isobutyrate), or fibrinogen.

8. The method of claim 7, wherein the three-dimensional scaffolding material is collagen used at a concentration of about 0.5 mg/ml to about 5.0 mg/ml.

9. The method of claim 8, wherein the collagen is native type I collagen.

10. The method of claim 1, wherein the cell culture medium of the adding step is DMEM and about 20% ES qualified fetal bovine serum and the three-dimensional scaffolding material is collagen at a concentration of about 0.4 mg/ml to about 1.0 mg/ml.

11. The method of claim 1, further comprising the step of inducing differentiation of the embryoid bodies to produce fibroblasts after the growing step.

12. The method of claim 11, wherein the inducing step comprises adding a cytokine to the three-dimensional embryoid body culture.

13. The method of claim 12, wherein the cytokine is vascular endothelial growth factor (VEGF); vascular permeability factor (VPF); members of the fibroblast growth factor family (FGF); members of the interleukin family (IL-1α, and -1β, -2, -3, -4, -5, -6, -7, -8, -9, -10, -11, -12, -13, -14, -15, -16, -17 or -18); epidermal growth factor (EGF); platelet-derived growth factor (PDGF); platelet-derived endothelial cell growth factor (PD-ECGF); transforming growth factors alpha and beta (TGF-α, TGF-β); tumor necrosis factor alpha (TNF α); hepatocyte growth factor (HGF); granulocyte-macrophage colony stimulating factor (GMCSF); insulin growth factor-1 (IGF-1); angiogenin; angiotropin; fibrin, nicotinamide; macrophage inflammatory protein (MIP); macrophage migration inhibiting factor (MIF); granulocyte stimulating factor (GCSF); macrophage stimulating factor (MCSF); endothelial cell growth factor (ECGF); members of the interferon family (IFNs); members of the insulin-like growth factor family (IGF-I and IGF-II); nerve growth factor (NGF); members of the neurotrophin family (NTs); members of the selectin family; intercellular adhesion molecule (ICAM); platelet vascular cell adhesion molecule (PECAM); vascular cell adhesion moleculre (VCAM); calcitonin, mediators, hormones or hirudin.

14. The method of claim 13, wherein the cytokine is transforming growth factor beta (TGF-β); fibroblast growth factor (FGF); or interleukin 4 (IL-4).

15. The method of claim 12, wherein the inducing step further comprises adding a cell culture medium comprising about 2% ES qualified fetal bovine serum.

16. The method of claim 11, further comprising the steps of;
isolating the differentiated cells from the three-dimensional scaffolding material; and
culturing the differentiated cells in monolayer culture;
after the inducing step.

17. The method of claim 16, wherein the isolating step is performed by digesting the three-dimensional scaffolding material and by centrifugation.

18. The method of claim 16, wherein the monolayer culture includes a culture medium of knock out DMEM and about 10% ES qualified fetal bovine serum.

19. The method of claim 12, wherein the inducing step includes adding FGF, TGF-b1 or IL-4 to the medium.

20. A differentiated fibroblast cultured by the method of claim 16.

21. A method of screening a compound for activity or cytotoxicity comprising combining the compound with the differentiated cell of claim 16 and determining any activity or cytotoxicity of the compound.

22. An embryoid body cultured by the method of claim 1.
